SEQUENCE LISTING (2 sequences)

SEQ ID NO: 1 - protein, 291 aa, Mus musculus
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 2 - DNA, 1461 bp, Artioposthia triangulata
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

(19) United States
(12) Patent Application Publication
(10) Pub. No.: US 2018/0223255 A1
(43) Pub. Date: Aug. 9, 2018

(54) Activation and Expansion of T Cells

(71) Applicant: Innovative Cellular Therapeutics Co., Ltd., Shanghai (CN)

(72) Inventors: Zhao Wu, Shanghai (CN); Lei Xiao, Shanghai (CN); Chengfei Pu, Shanghai (CN)

(73) Assignee: Innovative Cellular Therapeutics Co., Ltd., Shanghai (CN)

(21) Appl. No.: 15/939,402

(22) Filed: Mar. 29, 2018

Related U.S. Application Data
(63) Continuation of application No. PCT/CN2016/101441, filed on Oct. 8, 2016.
(60) Provisional application No. 62/238,894, filed on Oct. 8, 2015.

(51) Int. Cl. C12N 5/0783; A61K 35/17

(52) U.S. Cl. CPC C12N 5/0636; C12N 2510/00; C12N 2501/599; A61K 35/17

(57) ABSTRACT

The present disclosure relates to compositions, methods, and systems for selectively activating and/or expanding T cells for use in therapy. For example, the method may include contacting a population of T cells with an agent capable of binding an extracellular domain of a chimeric antigen receptor (CAR) that is expressed on the surface of the population of T cells.

### CROSS-REFERENCE TO RELATED PATENT APPLICATIONS

This application is a Continuation Application of International Patent Application No. PCT/CN2016/101441, filed on Oct. 8, 2016, which claims priority to U.S. Provisional Application No. 62/238,894, filed on Oct. 8, 2015, titled “Activation and expansion of T cells,” which are hereby incorporated by reference in their entirety.

### SEQUENCE LISTING INFORMATION

A computer readable textfile, entitled Activation and Expansion of T Cells “SDS1-0013US-Sequence Listing.txt,” created on or about Mar. 22, 2018, with a file size of about 8.00 KB, contains the sequence listing for this application and is hereby incorporated by reference in its entirety.

### TECHNICAL FIELD

The present disclosure relates to genetic engineering and medicine, in particular to compositions, methods, and systems for T cell activation and expansion.

### BACKGROUND

Chimeric antigen receptor T lymphocytes (i.e., CAR-T cells) identify tumor-specific markers and play a direct role in killing tumor cells. Since the first generation of CAR molecules was constructed, T cell expressing various CAR molecules have been widely used for treating diseases (e.g., cancers). One of the challenges of CAR-T based therapy is the development of efficient technologies and cost-effective clinical manufacturing platforms to allow safe and effective therapeutic uses.

### SUMMARY

Embodiments herein relate to compositions, methods, and systems for selectively activating and/or expanding T cells for use in therapy.

Some embodiments relate to a method of selectively activating a population of T cells for use in therapy. The method may include contacting the population of T cells with an agent capable of binding an extracellular domain of a CAR that is expressed on the surface of the population of T cells.

Some embodiments relate to a method of selectively ex vivo activating a population of T cells for use in therapy. The method may include contacting the population of T cells with an agent to activate the population of T cells that express a CAR to release IFN gamma. For example, the agent may include an extracellular domain of any antigen, for example, human CD19, and the CAR may include a CD19 antigen binding domain, a transmembrane domain, CD3-zeta domain and a costimulatory signaling domain.

Some embodiments relate to a method of selectively ex vivo activating a population of T cells for use in therapy. In some embodiments, the method may include contacting the population of T cells with an antibody that is immobilized on a solid surface. The antibody is capable of activating T cells that are to be transferred. In certain embodiments, a nucleic acid sequence encoding a CAR may be then transferred to the population of T cells. After the CAR expresses on the surface of the population of T cells, the solid surface may be removed from the population of T cells after the transferring, and the population of T cells may be then contacted with an agent to provide sustained activation of the population of T cells that express the CAR and to expand the population of T cells to a certain number suitable for use in therapy. In some instances, the agent may include an extracellular domain of human CD19, and/or the CAR may include a CD19 antigen binding domain, a transmembrane domain, CD3-zeta domain and a costimulatory signaling domain.

In some embodiments, the method may further include, prior to contacting the population of T cells with the agent, contacting the population of T cells with an anti-CD3 antibody that is immobilized on a solid surface for a time period. In some embodiments, the time period can be an hour to 48 hours. In other embodiments, the time period could be can be one day to two days. Subsequently, a nucleic acid sequence encoding the CAR may be transferred to the contacted population of T cells, and the solid surface from the population of T cells may be then removed.

In some embodiments, the solid surface is further attached with an anti-CD28 antibody.

In some embodiments, the method may further include collecting peripheral blood mononuclear cells (PBMCs) from a subject and selecting CD3⁺ cells from the PBMCs. In certain embodiments, the PBMCs may be mixed with a group of antibodies to allow the group of antibodies to bind target cells. In these instances, the group of antibodies does not include CD3 antibodies. The target cells may be then removed from the PBMCs to obtain a solution containing the CD3⁺ cells. For example, the group of antibodies may include at least one of CD14, CD15, CD16, CD19, CD34, CD36, CD56, CD123, or CD235a.

In some embodiments, the CAR may include an antigen binding domain, a transmembrane domain, and a costimulatory signaling domain; and the agent may include an extracellular domain of an antigen for binding to the antigen binding domain of the CAR.

In some embodiments, the extracellular domain of the CAR is a CD19 antigen binding domain. For example, the agent is a CD19 antigen, which includes the extracellular domain of human CD19. In certain embodiments, the CD19 antigen may include the amino acid sequence of SEQ ID NO: 1.

In some embodiments, the CAR may include a CD19 antigen binding domain, a transmembrane domain, CD3-zeta domain, and a costimulatory signaling domain.

In some embodiments, the agent is attached to a surface. In certain embodiments, the surface is at least one of a biocompatible, biodegradable, non-biodegradable, natural, or synthetic surface. For example, the surface is a magnetic bead. In certain embodiments, the bead is capable of activating the population of T cells expressing the CAR to release IFN gamma. In some embodiments, the agent is an extracellular domain of a tumor antigen attached to a bead without further being attached to an antibody (such as the 4-1BB antibody) or a 4-1BB binding fragment thereof. In other embodiments, the surface of the bead is further attached with a 4-1BB antibody or a 4-1BB binding fragment thereof.

In some embodiments, the population of T cells is ex vivo expanded to sufficient numbers for use in therapy. In certain embodiments, the population of T cells is expanded to about 100-fold of the original T cell population. In certain embodiments, the population of T cells is expanded to about 100,000-fold of the original T cell population.

Some embodiments relate to a composition including a magnetic particle conjugated with a CD19 antigen that includes an extracellular domain of human CD19. For example, the magnetic particle is capable of selectively ex vivo activating T cells that express a CAR for use in therapy. The CAR includes a CD19 antigen binding domain, a transmembrane domain, CD3-zeta domain, and a costimulatory signaling domain.

In some embodiments, the T cells have been collected from PBMCs and previously activated using an anti-CD3 antibody or CD3 binding fragment thereof.

In some embodiments, the magnetic particle is capable of activating the T cells of which express the CAR to release IFN gamma.

This Summary is provided to introduce a selection of concepts in a simplified form that is further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 is a schematic diagram illustrating an exemplary process for activation and expansion of T cells.

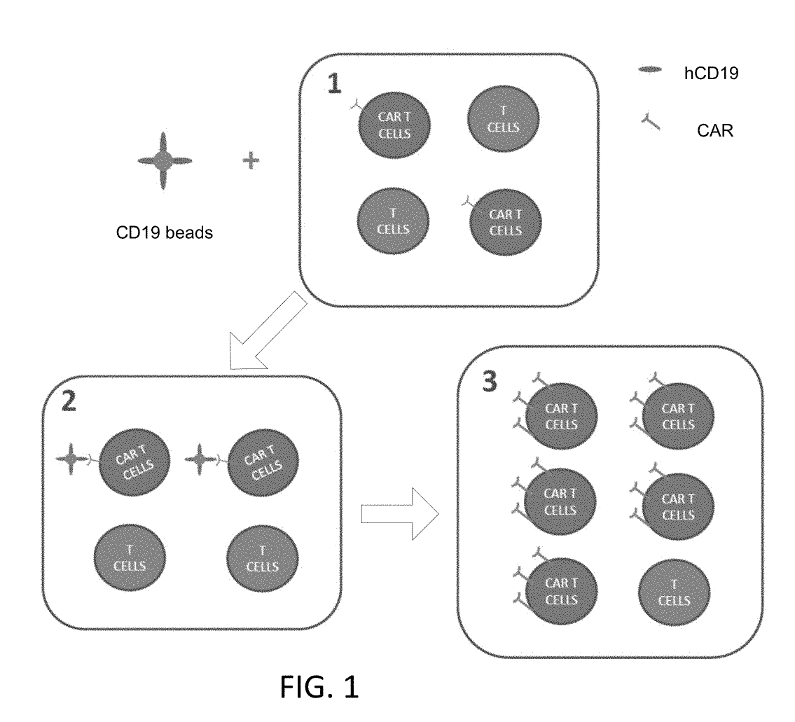

FIG. 2 is a schematic diagram illustrating exemplary structures of beads in accordance with embodiments of the present disclosure.

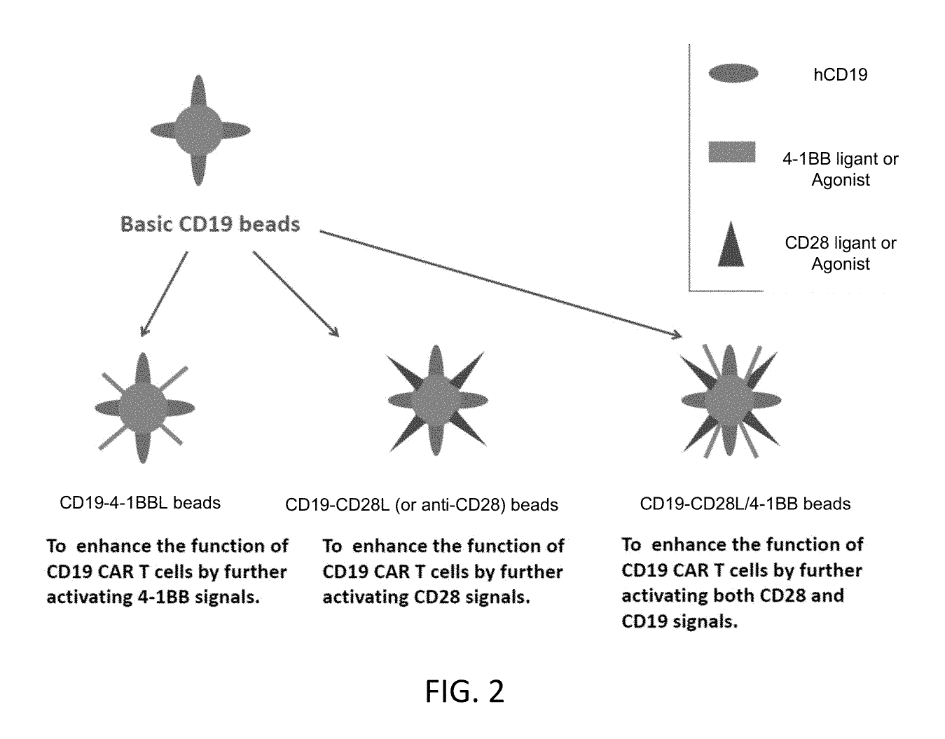

FIG. 3 is a flow cytometric analysis diagram indicating preparation of beads conjugated with a CD19 antigen or a CD22 antigen.

FIG. 4 is a flow cytometric analysis diagram indicating preparation of anti-CD19 CAR-T cells.

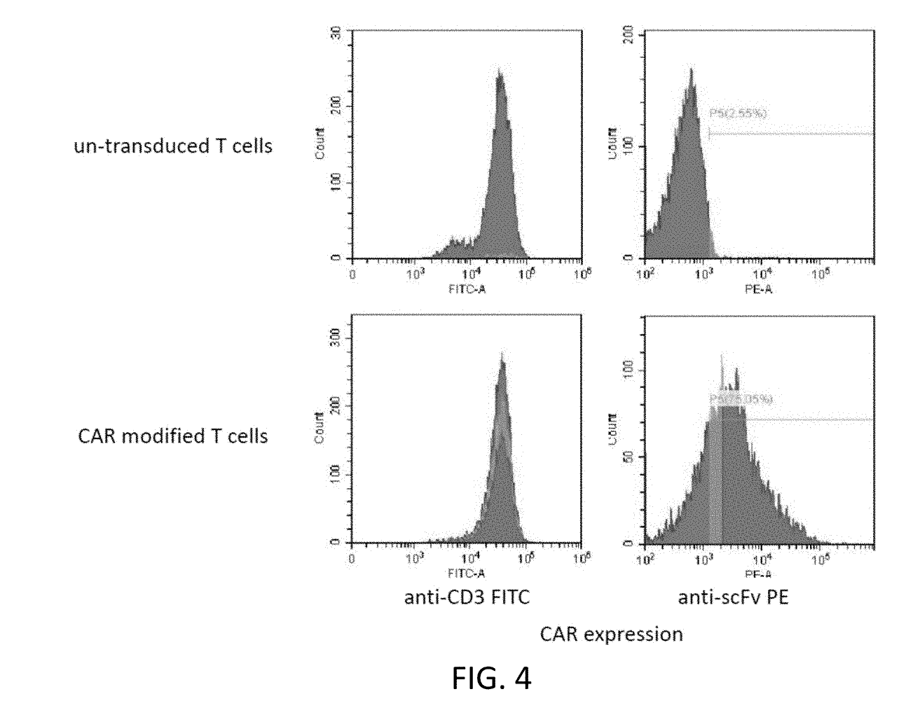

FIG. 5 is a flow cytometric analysis diagram indicating that beads conjugated with a CD19 antigen activate T cells expressing CARs.

FIG. 6 is a diagram showing T cell proliferation stimulated by CD19 beads.

FIG. 7 includes diagrams showing parameters associated with T cells in response to exposure to beads conjugated with a CD19 antigen.

FIG. 8 includes exemplary flow cytometric analysis diagrams showing measurement of some parameters illustrated in FIG. 7.

FIG. 9 includes images showing functionalities of T cells that are contacted with beads conjugated with a CD19 antigen.

FIG. 10 is a flow cytometric analysis diagram showing a selection of T cells in accordance with embodiments of the present disclosure.

FIG. 11 includes schematic diagrams showing T cell growth rates under different selection methods.

FIG. 12 is a schematic diagram showing T cell transduction rates under different selection methods.

### DETAILED DESCRIPTION

### Overview

Ex vivo expansion of primary T cells requires continuous or sustained activation to maintain a good condition for therapeutic uses. For example, beads conjugated with anti-CD3 and anti-CD28 antibodies are widely used for T cell activation. Under conventional techniques, isolated T cells are contacted with these beads until the population of T cells reaches a certain number suitable for therapeutic uses. However, prolonged anti-CD28 T cell stimulation may cause T cell exhaustion which is a state of T cell dysfunction, T cell exhaustion prevents optimal control of infection and tumors.

Embodiments herein utilize beads conjugated with a CD19 antigen to selectively activate and/or expand T cells that express an anti-CD19 CAR. The CD19 antigen may activate the T cells via signals mediated by, for example, 4-1BB and/or CD3-zeta. Accordingly, by avoiding prolonged stimulation of anti-CD28, the embodiments may reduce or delay T cell exhaustion.

### Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a T cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and includes monoclonal antibodies (including full length monoclonal antibodies), multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

“Antibody fragments” comprise a portion of a full length antibody, generally the antigen binding or variable region of the antibody. Examples of antibody fragments include Fab, Fab′, F(ab′)2, and Fv fragments; diabodies; linear antibodies; single-chain antibody molecules; and multi-specific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. k and λ light chains refer to the two major antibody light chain isotypes.

The term “synthetic antibody” as used herein, refers to an antibody which is generated using recombinant DNA technology, such as, for example, an antibody expressed by a bacteriophage as described herein. The term may also be construed to refer to an antibody which has been generated by the synthesis of a DNA molecule encoding the antibody and which DNA molecule expresses an antibody protein, or an amino acid sequence encoding the antibody, wherein the DNA or amino acid sequence has been obtained using synthetic DNA or amino acid sequence technology which is available and well known in the art.

The term “antigen” as used herein, refers to a molecule that provokes an immune response, which may involve either antibody production, or the activation of specific immunologically-competent cells, or both. Antigens may include any macromolecule, including virtually all proteins or peptides, or molecules derived from recombinant or genomic DNA. For example, DNA including a nucleotide sequence or a partial nucleotide sequence encoding a protein that elicits an immune response; and therefore, encodes an “antigen” as that term is used herein. Further, an antigen need not be encoded solely by a full length nucleotide sequence of a gene. Furthermore, an antigen can be generated, synthesized or derived from a biological sample including a tissue sample, a tumor sample, a cell or a biological fluid.

The term “anti-tumor effect” as used herein, refers to a biological effect associated with a decrease in tumor volume, a decrease in the number of tumor cells, a decrease in the number of metastases, an increase in life expectancy of a subject having tumor cells, or amelioration of various physiological symptoms associated with the cancerous condition. An “anti-tumor effect” can also be manifested by the ability of the peptides, polynucleotides, cells, and antibodies of the disclosure in the prevention of the occurrence of tumor in the first place.

The term “auto-antigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the subject.

“Allogeneic” is used to describe a graft derived from a different subject of the same species.

“Xenogeneic” is used to describe a graft derived from a subject of a different species.

The term “cancer” as used herein is defined as a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer et al.

Throughout this specification, unless the context requires otherwise, the words “comprise,” “includes” and “including” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

“Co-stimulatory ligand,” includes a molecule on an antigen presenting cell (e.g., an APC, dendritic cell, B cell, et al.) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including proliferation, activation, differentiation, et al. A co-stimulatory ligand can include CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

A “co-stimulatory signal” refers to a signal, which in combination with a primary signal, such as TCR/CD3 ligation, leads to T cell proliferation and/or upregulation or down regulation of key molecules.

As used herein, the terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. As used herein, a “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “effective” means adequate to accomplish a desired, expected, or intended result. For example, an “effective amount” may be an amount of a compound sufficient to produce a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

With regard to polynucleotides, the term “exogenous” refers to a polynucleotide sequence that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides, the term “endogenous” or “native” refers to naturally-occurring polynucleotide sequences that may be found in a given wild-type cell or organism. Also, a particular polynucleotide sequence that is isolated from a first organism and transferred to the second organism by molecular biological techniques is typically considered an “exogenous” polynucleotide with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” as used herein is defined as the transcription and/or translation of a particular nucleotide sequence driven by its promoter.

“Expression vector” refers to a vector including a recombinant polynucleotide including expression control sequences operatively linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous, then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

By “isolated” is meant a material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” et al. are used interchangeably herein, and refer to any animal, amenable to the methods described herein. In certain non-limiting embodiments, the patient, subject or individual is a human. In some embodiments, the term “subject” is intended to include living organisms in which an immune response can be elicited (e.g., mammals). Examples of subjects include humans, dogs, cats, mice, rats, and transgenic species thereof.

The recitation “polynucleotide” or “nucleic acid” as used herein designates mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “promoter” as used herein is defined as a DNA sequence recognized by the synthetic machinery of the cell or introduced synthetic machinery, required to initiate the specific transcription of a polynucleotide sequence. The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” means that one molecule recognizes and adheres to a particular second molecule in a sample or organism, but does not substantially recognize or adhere to other structurally unrelated molecules in the sample. By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

A “soluble receptor” is a receptor polypeptide that is not bound to a cell membrane. Soluble receptors are most commonly ligand-binding receptor polypeptides that lack transmembrane and cytoplasmic domains. Soluble receptors may include additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis. Soluble receptor polypeptides are said to be substantially free of transmembrane and intracellular polypeptide segments when they lack sufficient portions of these segments to provide membrane anchoring or signal transduction, respectively.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By the term “stimulation,” is meant a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures et al.

A “stimulatory molecule” refers to a molecule on a T cell that specifically binds to a cognate stimulatory ligand present on an antigen presenting cell.

A “stimulatory ligand” refers to a ligand that when present on an antigen presenting cell (e.g., an APC, a dendritic cell, a B-cell, et al.) can specifically bind with a cognate binding partner (referred to herein as a “stimulatory molecule”) on a T cell, thereby mediating a primary response by the T cell, including activation, initiation of an immune response, proliferation, et al. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to a cell that has been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

To “treat” disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which includes an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, et al. Examples of viral vectors include, but are not limited to, adenoviral vectors, adeno-associated virus vectors, retroviral vectors, et al. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present disclosure relates to isolated nucleic acid sequences, vectors including the isolated nucleic acid sequences, cells including the isolated nucleic acid sequences and methods of treating cancer using these cells.

### Compositions, Therapeutic Application and Preparation Methods Thereof

Embodiments herein relate to compositions, methods, and systems for selectively activating and/or expanding T cells for use in therapy.

Some embodiments relate to a method of selectively activating a population of T cells for use in therapy. The method may include contacting a population of T cells with an agent capable of binding an extracellular domain of a CAR that is expressed on the surface of the population of T cells.

Some embodiments relate to a method of selectively ex vivo activating a population of T cells for use in therapy. The method may include contacting the population of T cells with an agent to activate the population of T cells that express a CAR to release IFN gamma.

In some embodiments, the agent is an antigen that binds the extracellular domain of a CAR. In certain embodiments, the agent comprises an extracellular domain of at least one of Epidermal growth factor Receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation Protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, or CD4. For example, the agent may include an extracellular domain of human Cluster of differentiation 19 (CD19), and the CAR may include a CD19 antigen binding domain, a transmembrane domain, CD3-zeta domain and a costimulatory signaling domain.

Some embodiments relate to a method of selectively ex vivo activating a population of T cells for use in therapy. In some embodiments, the method may include contacting the population of T cells with an antibody that is immobilized on a solid surface. The antibody is capable of activating T cells that are to be transferred. In certain embodiments, a nucleic acid sequence encoding a CAR may be then transferred to the population of T cells. After the CAR expresses on the surface of the population of T cells, the solid surface may be removed from the population of T cells after the transferring, and the population of T cells may be then contacted with an agent to provide sustained activation of the population of T cells that express the CAR and to expand the population of T cells to a certain number suitable for the therapy uses. In some instances, the agent may include an extracellular domain of human CD19, and/or the CAR may include a CD19 antigen binding domain, a transmembrane domain, CD3-zeta domain and a costimulatory signaling domain.

CD19 is about 90 kDa, as identified, for example, by the HD237 or B4 antibody (Kiesel et al., Leukemia Research II, 12:1119 (1987)). CD19 is found on cells throughout differentiation of B-lineage cells from the stem cell stage through terminal differentiation into plasma cells, including but not limited to, pre-B cells, B cells (including naïve B cells, antigen-stimulated B cells, memory B cells, plasma cells, and B lymphocytes) and follicular dendritic cells. CD19 is also found on B cells in the human fetal tissue. In some embodiments, the CD19 antigen targeted by the antibodies of the present disclosure is a human CD19 antigen.

In some embodiments, a CD19 antigen may include a portion or all of the extracellular domain of human CD19. In some instances, the CD19 antigen may include a signal peptide of human CD19 and/or the extracellular domain of human CD19. For example, the CD19 antigen may include about 283 amino acids (including the signal peptide and the extracellular domain of human CD19) and is predicted a molecular mass of 31.6 kDa; in SDS-PAGE under reducing conditions, this CD19 antigen migrates as an approximately 47 kDa band due to glycosylation. In certain embodiments, the CD19 antigen comprises an amino acid sequence of SEQ ID NO: 1.

In some embodiments, the CAR may include an antigen binding domain, a transmembrane domain, CD3-zeta domain, and a costimulatory signaling domain; and the agent may include an extracellular domain of an antigen that binds the antigen binding domain of the CAR.

In some embodiments, the extracellular domain of the CAR is a CD19 antigen binding domain. For example, the agent is a CD19 antigen, which includes the extracellular domain of human CD19. In certain embodiments, the CD19 antigen may include the amino acid sequence of SEQ ID NO: 1.

In some embodiments, the CAR may include a CD19 antigen binding domain, a transmembrane domain, CD3-zeta domain, and a costimulatory signaling domain.

In some embodiments, the agent is attached to a surface. In certain embodiments, the surface is at least one of a biocompatible, biodegradable, non-biodegradable, natural, or synthetic surface. For example, the surface is a magnetic bead. In certain embodiments, the bead is capable of activating the population of T cells expressing the CAR to release IFN gamma. In some embodiments, the agent is an extracellular domain of a tumor antigen attached to a bead without further being attached to an antibody (such as the 44-1BB antibody) or a 4-1BB binding fragment thereof. In other embodiments, the surface of the bead is further attached with a 4-1BB antibody or a 4-1BB binding fragment thereof.

In some embodiments, the population of T cells is ex vivo expanded to sufficient numbers for use in therapy. In certain embodiments, the population of T cells is expanded to about 100-fold of the original T cell population. In certain embodiments, the population of T cells is expanded to about 100,000-fold of the original T cell population.

Some embodiments relate to a composition including a magnetic particle conjugated with a CD19 antigen that includes a portion or the whole of an extracellular domain of human CD19 (CD19 beads). For example, the magnetic particle and the CD19 antigen are fused together such that the conjugated magnetic particle is capable of selectively ex vivo activating T cells that express a CAR for use in therapy. The CAR includes a CD19 antigen binding domain, a transmembrane domain, CD3 Zeta domain, and a costimulatory signaling domain (e.g., 4-1BB and/or CD28). In other embodiments, the CD19 antigen may be conjugated with a carrier such as a solid surface other than beads or cells, and the carrier is then capable of selectively ex vivo activating T cells that express anti-CD19 CAR.

In some embodiments, the T cells have been collected from PBMCs and previously activated using an anti-CD3 antibody or CD3 binding fragment thereof. For example, primary activation of T cells may be implemented by co-culturing primary T cells isolated from PBMCs with beads conjugated with anti-CD3 beads. The activated T cells may be transferred with virus containing a CAR. After the CAR is expressed on the surface of the T cells (e.g., two days after transduction), the anti-CD3 antibody or CD3 binding fragment thereof may be removed from the culture containing the T cells. For example, the anti-CD3 antibody or CD3 binding fragment thereof may be conjugated with beads, and the beads may be removed from the culture. To expand T cells, sustained activation may be then implemented by co-culturing the T cells with beads conjugated with a CD19 antigen. In some embodiments, the beads are conjugated with the CD19 antigen, and therefore is capable of activating the T cells of which express the CAR to release IFN gamma.

In some embodiments, the method may adopt negative selection by removing CD3 negative cells from PBMCs and therefore obtain CD3 positive cells including I cells Surprisingly, the embodiments have higher transduction efficiency as compared to the conventional techniques as described below.

Currently, techniques for preparation of CAR-T cells may be divided into three approaches. For the first approach, CAR-T cells are obtained directly from PBMCs. For example, apheresis density gradient centrifugation is performed to obtain PBMCs, which are then incubated with IL2 and CD3 agonist to prepare transducted CAR-T cells. However, because the transduced cells include not only T cells but also monocytes as well as other cells, transduction efficiency of this approach is low.

For the second approach, apheresis density gradient centrifugation is performed to obtain PBMCs from which monocytes are then removed. The remaining cells are transduced to obtain CAR-T cells. Still, the transduction efficiency of the second approach is low since the transduced cells include various other cells in addition to T cells.

For the third approach, apheresis density gradient centrifugation is performed to obtain PBMCs, which are then incubated with beads coated with CD3/CD28 antibodies to obtain T cells for transduction. While enriching T cells using CD3/CD28, the resulting cells under the third approach still include various other cells. Therefore, transduction efficiency is still low, and functions of T cells obtained using this approach is unstable.

In some embodiments, the method may further include, prior to contacting the population of T cells with the agent, contacting the population of T cells with an anti-CD3 antibody that is immobilized on a solid surface for a time period. In some embodiments, the time period could be an hour to 48 hours. In other embodiments, the time period could be could be one day to two days. Then, a nucleic acid sequence encoding the CAR may be transferred to the contacted population of T cells, and the solid surface from the population of T cells may be then removed. In some embodiments, the solid surface is further attached with an anti-CD28 antibody.

For example, peripheral blood mononuclear cells (PBMCs) may be collected from a subject, CD3⁺ cells from the PBMCs may be obtained, and the CD3⁺ cells may be activated via the primary signal pathway (e.g., binding of a TCR/CD3 complex) to obtain activated T cells. A nucleic acid sequence encoding the CAR may be transferred to the activated T cells to obtain CAR-T cells. In certain embodiments, the method may include mixing the PBMCs with a group of antibodies to allow the group of antibodies to bind target cells and removing the target cells from the PBMCS to obtain a solution containing the CD3⁺ cells. In these instances, the group of antibodies does not include CD3 antibodies. For example, the group of antibodies may include at least one of CD14, CD15, CD16, CD19, CD34, CD36, CD56, CD123, or CD235a. In some instances, the transduction rate of the transferring of the activated T cells is at least 50% as measured on day 5, 6, or 7 after the transferring.

T cells may be activated using various methods including certain embodiments of the present disclosure. In some embodiments, T cells may be activated by co-culturing T cells with antibodies (e.g., antibodies of CD3 and/or CD28). In some embodiments, T cells may be co-cultured with beads (e.g., magnetic beads) conjugated with antibodies (e.g., antibodies of CD3 and/or CD28). In some embodiments, various cytokines (e.g., IL2, IL7, IL15) may be used to activate T cells. In certain embodiments, a combination of cytokines and antibodies may be used to activate T cells. In certain embodiments, a combination of cytokines and beads may be used to activate T cells.

CARs are molecules generally including an extracellular and intracellular domain. The extracellular domain includes a target-specific binding element. The intracellular domain (e.g., cytoplasmic domain) includes a costimulatory signaling region and a zeta chain portion. The costimulatory signaling region refers to a portion of the CAR including the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may include up to 300 amino acids, preferably 10 to 100 amino acids, and most preferably 25 to 50 amino acids.

In some embodiments, the target-specific binding element of the CAR in the present disclosure may recognize a tumor antigen. Tumor antigens are proteins that are produced by tumor cells that elicit an immune response, particularly T-cell mediated immune responses. Tumor antigens are well known in the art and include, for example, a glioma-associated antigen, carcinoembryonic antigen (CEA), (β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1α, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor a 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor. Examples of viruses that cause human cancers include human papillomavirus, hepatitis B, hepatitis C virus, Epstein-Barr virus, human T-lymphotopic virus, Kaposi's sarcoma-associated herpesvirus, and Merkel cell polyomavirus.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The embodiments of the present disclosure further relate to vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from oncoretroviruses such as murine leukemia viruses in that they can transduce non-proliferating cells, such as hepatocytes. They also have the added advantage of low immunogenicity.

The embodiments further relate to methods of treating a patient for illness including administering to the patient an effective amount of the engineered cells of the present disclosure. Various illnesses can be treated according to the present methods including cancer, such as ovarian carcinoma, breast carcinoma, colon carcinoma, glioblastoma multiforme, prostate carcinoma and leukemia. In some embodiments, the method includes administering to a human patient a pharmaceutical composition including an antitumor effective amount of a population of human T cells, wherein the human T cells of the population include human T cells that comprises the nucleic acid sequence as described in the present disclosure.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases).

Generally, the cells activated and expanded as described herein may be utilized in the treatment and prevention of diseases that arise in individuals who are immunocompromised. In particular, the engineered cells of the present disclosure are used in the treatment of cancer. In certain embodiments, the cells of the present disclosure are used in the treatment of patients at risk for developing cancer. Thus, the present disclosure provides methods for the treatment or prevention of cancer comprising administering to a subject in need thereof, a therapeutically effective amount of the engineered T cells of the present disclosure.

The engineered T cells of the present disclosure may be administered either alone or as a pharmaceutical composition in combination with diluents and/or with other components such as IL-2 or other cytokines or cell populations. Briefly, pharmaceutical compositions of the present disclosure may include a target cell population as described herein, in combination with one or more pharmaceutically or physiologically acceptable carriers, diluents or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have apheresis performed), activate T cells therefrom according to the present disclosure, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, may select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i. v.) injection, or intraperitoneally. In one embodiment, the T cell compositions of the present disclosure are administered to a patient by intradermal or subcutaneous injection. In another embodiment, the T cell compositions of the present disclosure are preferably administered by i.v. injection. The compositions of T cells may be injected directly into a tumor, lymph node, or site of infection.

In certain embodiments of the present disclosure, cells activated and/or expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, irrimunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p7056 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In other embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In further embodiments, the cell compositions of the present disclosure are administered following B-cell ablative therapy such as agents that react with CD20, e.g., Rituxan. For example, in embodiments, subjects may undergo standard treatment with high dose chemotherapy followed by peripheral blood stem cell transplantation. In certain embodiments, following the transplant, subjects receive an infusion of the expanded immune cells of the present disclosure. In additional embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766, incorporated by reference in its entirety).

Additional information on the methods of cancer treatment using engineer T cells is provided in U.S. Pat. No. 8,906,682, incorporated by reference in its entirety.

Acceptable in vitro or animal models for immunotherapy include: leukemia mouse model, in vitro cell killing experiments, drug dose and route of administration including relevant animal models and humans. Selected cells are modified to obtain CAR-T cells, which may be used for intravenous transfusion, according to dose 10⁵˜10⁷/kg body weight.

### EXAMPLES

The present disclosure is further described by reference to the following examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teaching provided herein.

### Example 1

### Preparation of Beads Conjugated with CD19 Extracellular Domain and CD22 Extracellular Domain

The CD19 antigen used was the human CD19 including the signal peptide and the extracellular domain. The 6 His tag (e.g., 6-histidine) was attached to the C terminal of the CD19 antigen. The CD19 antigen was expressed in HEK293E cells and purified afterward. Binding buffer was used after resuspension of the CD19 antigen, which was further conjugated with magnetic beads to obtain CD19 beads.

Magnetic beads were obtained from Life Technology® (Catalog NOs. 10103D). The magnetic beads were sepharose beads, the surface of which was conjugated with Co²⁺ ions. The Co²⁺ ions on the surface of the magnetic beads are capable of binding to the His tag, such that CD19 beads were prepared accordingly.

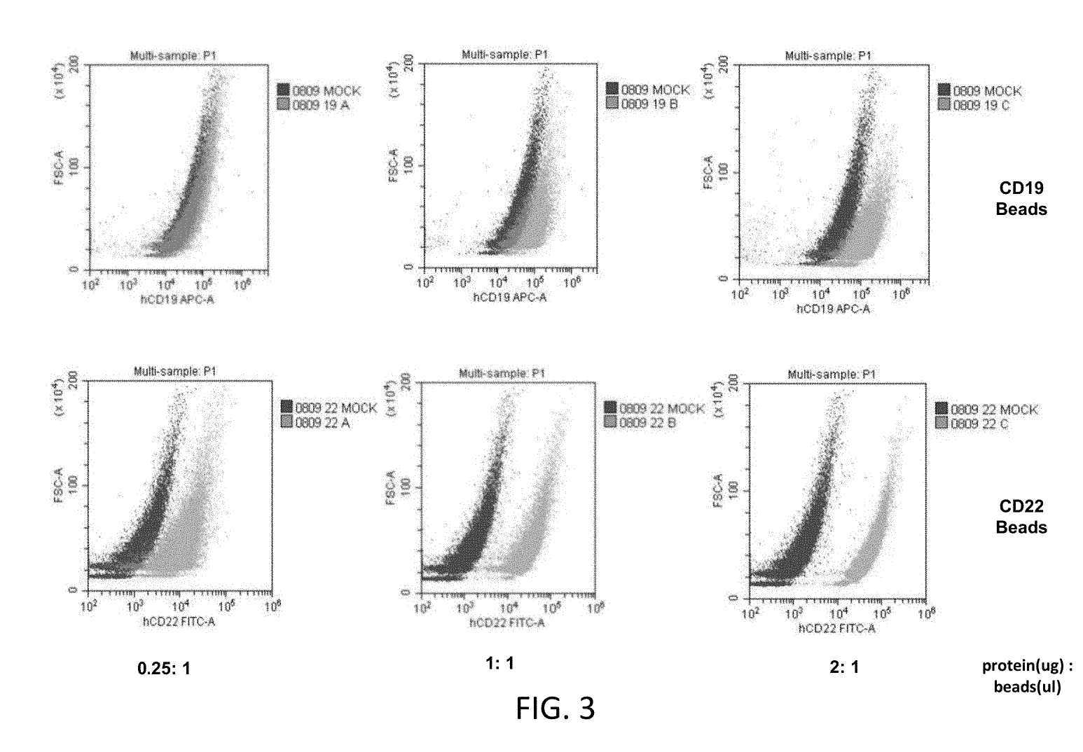

Peptides including CD19 and CD22 extracellular domain (ECD) were mixed with Co²⁺ magnetic beads. The C terminals of the CD19 and CD22 ECD contained His tags such that beads were conjugated with CD19 and CD 22 ECD. Different gradients (e.g., ratios between amounts of peptides and volumes of beads) were analyzed for testing conjugation of beads and peptides. Flow cytometric analysis was then performed to measure the binding efficiency, as illustrated in FIG. 3.

Lentiviral virus containing a nucleonic acid sequence (SEQ ID NO: 2) encoding an anti-CD19 CAR was transferred to primary T cells, which were isolated for flow cytometric analysis (see Chimeric Receptors Containing CD137 Signal Transduction Domains Mediate Enhanced Survival of T Cells and Increased Antileukemic Efficacy in vivo Molecular Therapy vol. 17 no. 8, 1453-1464 August 2009 incorporated herein by reference). The anti-CD19 CAR contained a 4-1BB signal transduction domain and a CD3-zeta domain.

### Example 2

### Activation of Anti-CD19 CAR-T Cells Stimulated by CD19 Beads

K562 cells, K562-CD19 cells (K562 cells expressing CD19), CD19 beads and CD22 beads were co-cultured with an equal amount NT (un-transduced T cell) or CAR-T cells for 24 hours, and flow cytometric analysis was performed to detect IFN gamma release.

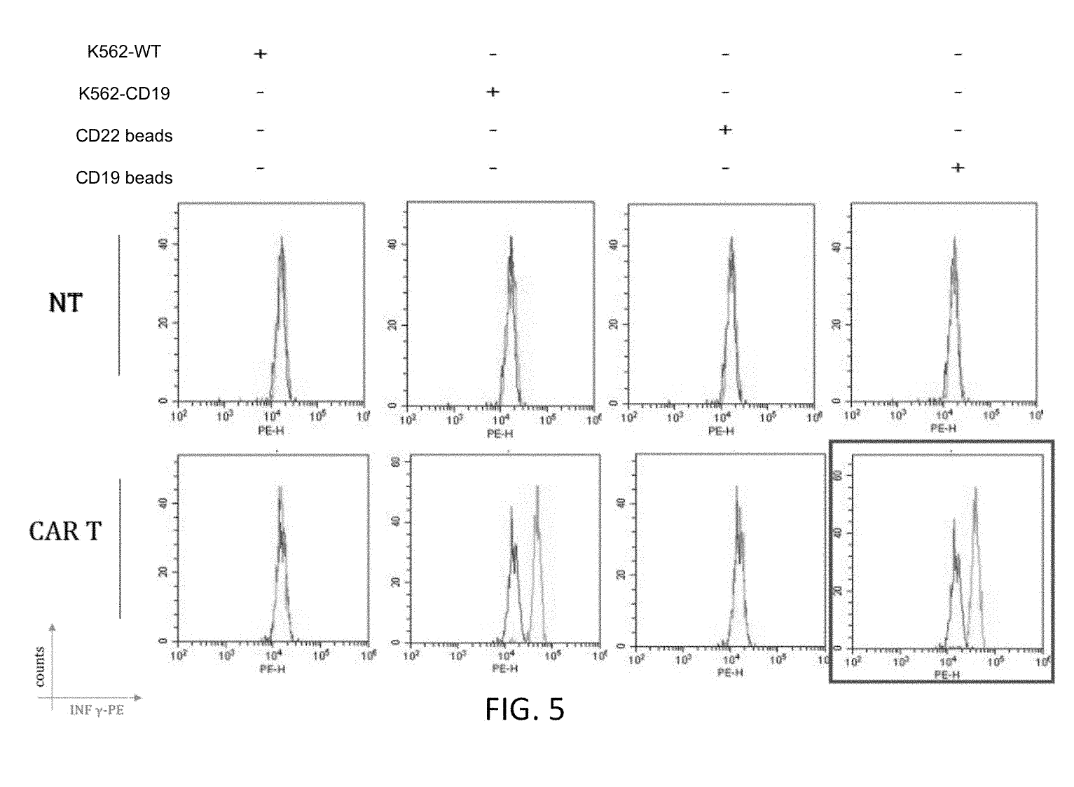

K562-wt and K562-CD19 represented negative and positive control, respectively, with respect to the ability to cause IFN gamma release. As illustrated in FIG. 5, CD22 beads failed to cause IFN gamma release, and CD19 beads caused IFN gamma release, as indicated by two peaks (box in FIG. 5). In the flow cytometry diagrams of FIG. 5, the horizontal axis represented the intensity of the release of IFN gamma.

### Example 3

### Expansion of T cells Stimulated by CD19 Beads

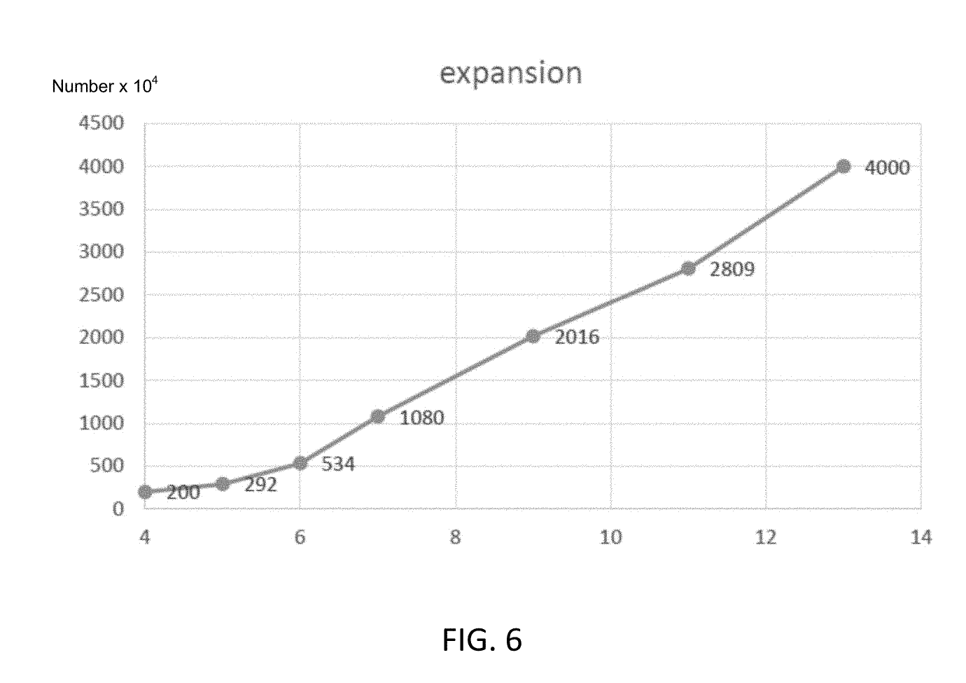

Primary T cells were isolated and contacted with beads conjugated with anti-CD3 and anti-CD28 on Day 1. On Day 2, the T cells were transferred with lentiviral virus containing a nucleic acid sequence (SEQ ID NO: 2) encoding an anti-CD19 CAR. On Day 4, the beads conjugated with anti-CD3 and anti-CD28 were removed from the culture, and CD19 beads were co-cultured with the T cells. T cell proliferation was measured from Day 6 and illustrated in FIG. 6, which shows that the CD19 beads successfully stimulated T cells to expand.

### Example 4

### Sustained Activation of T cells Mediated by CD19 Beads

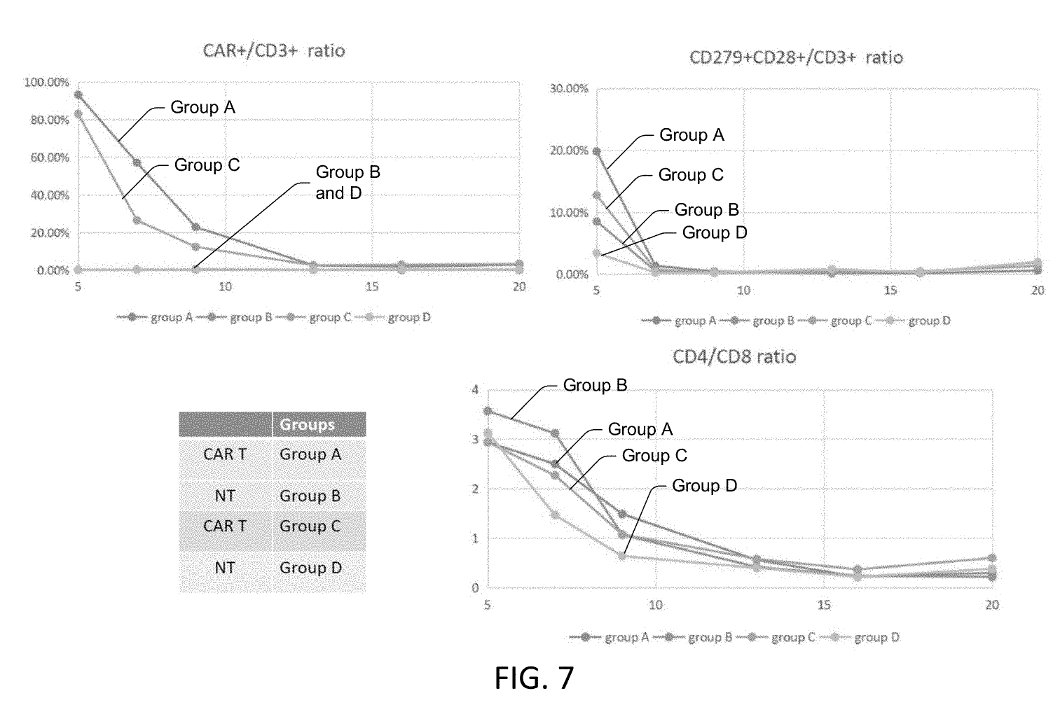

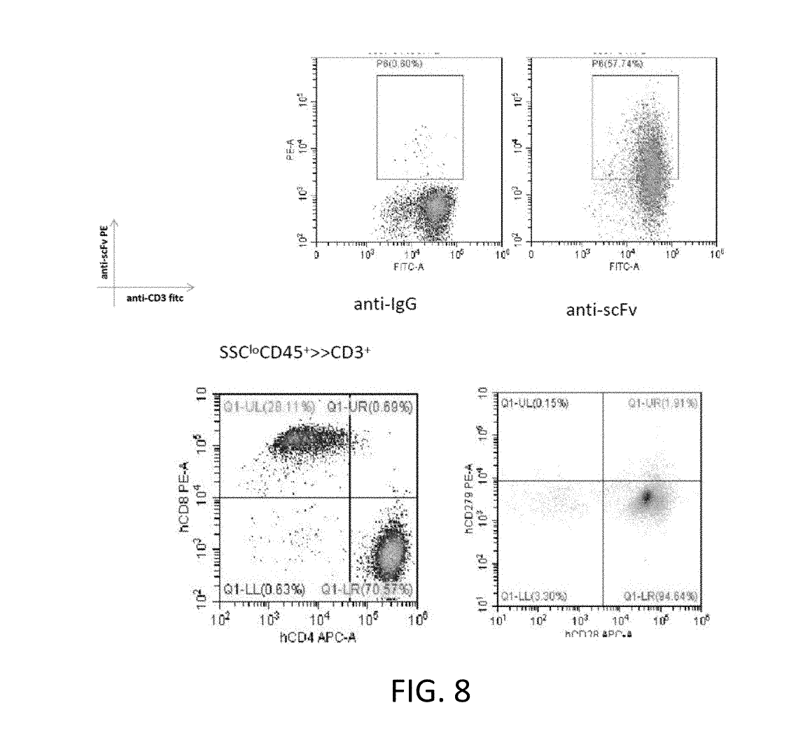

As illustrated in Table 1, several groups of primary T cells were isolated and contacted with beads conjugated with anti-CD3 and anti-CD28 on Day 1. On Day 2, the T cells were transferred with lentiviral virus containing a nucleonic acid sequence (SEQ ID NO: 2) encoding an anti-CD19 CAR. On Day 4, the beads conjugated with anti-CD3 and anti-CD28 were removed from the culture, and CD19 beads were co-cultured with the T cells (Groups C and D) for at least 3 days. As for Groups A and B, T cells were continuously cultured with the beads conjugated with anti-CD3 and anti-CD28. Various parameters were measured to determine whether T cells remained healthy and functional using flow cytometric analysis after Day 5, as illustrated in FIGS. 7-9. For Group C and D, beads conjugated with anti-CD3 (without anti-CD28) were also used, and similar results were obtained.

| | Groups | Day 1 | Day 2 | Day 4 |
|---|---|---|---|---|
| CAR-T | A | T cell isolation | Transduction | Cultured with ant-CD3 |
| | | and activation | | and anti-CD28 beads |
| NT | B | T cell isolation | No | Cultured with ant-CD3 |
| | | and activation | Transduction | and anti-CD28 beads |
| CAR-T | C | T cell isolation | Transduction | Removed previously |
| | | and activation | | added beads for |
| | | | | primary activation |
| | | | | and then cultured |
| | | | | with CD19 beads |
| NT | D | T cell isolation | No | Removed previously |
| | | and activation | Transduction | added beads for |
| | | | | primary activation |
| | | | | and then cultured |
| | | | | with CD19 beads |

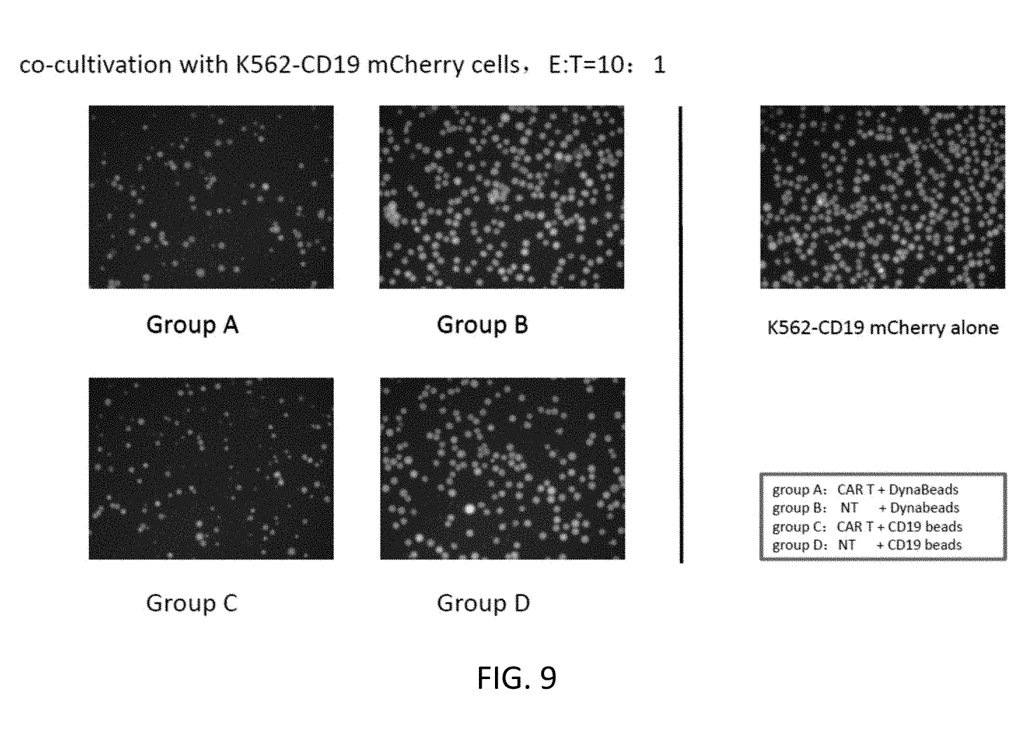

Cells from each of Groups A, B, C, and D were cultured with K562-CD19 mCherry cells. As indicated in FIG. 9, the cells from Groups A and C were capable of killing CD19 positive cells. These results show that sustained activation of T cells can be mediated by the CD19 beads and the population of T cells remains healthy and functional.

### Example 5

### Separation Methods (10 Ml)

A blood sample from a subject was diluted using DPBS. Apheresis density gradient centrifugation was performed to obtain PBMCs containing lymphocytes. MACS Buffer was used to rinsing PBMCs. Pan T Cell-Ab cocktail was mixed with PBMCs and incubated for 5 min. Pan T cell beads cocktail was added and incubated at 2-8 degrees for 10 min. LS columns were used to collectct CD3⁺ cells.

### Example 6

### Evaluation of the Potential Advantages of Isolating T Cells Using the Protocol of the Present Disclosure Against Conventional T Cell Isolation Protocols

Two commonly used T cell isolation protocols and two Dynabeads stimulation ratios were evaluated as follow. 1. Protocol of the present disclosure: Immune cells are isolated from whole blood using histopaque followed by positive selection through an affinity column. 2. Whole immune cells are isolated using histopaque. 3. Whole immune cells are isolated using histopaque, followed by stimulation using 3-times the number of magnetic beads. Evaluation parameters may include CD3 purity, T cell proliferation rate, Lentivirus transduction ratio, and/or T cell proliferation rate after transduction.

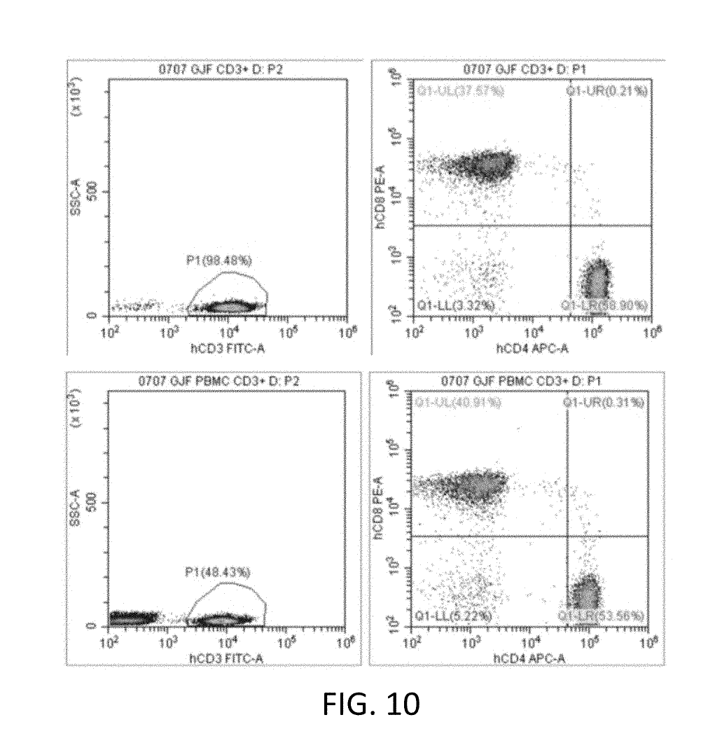

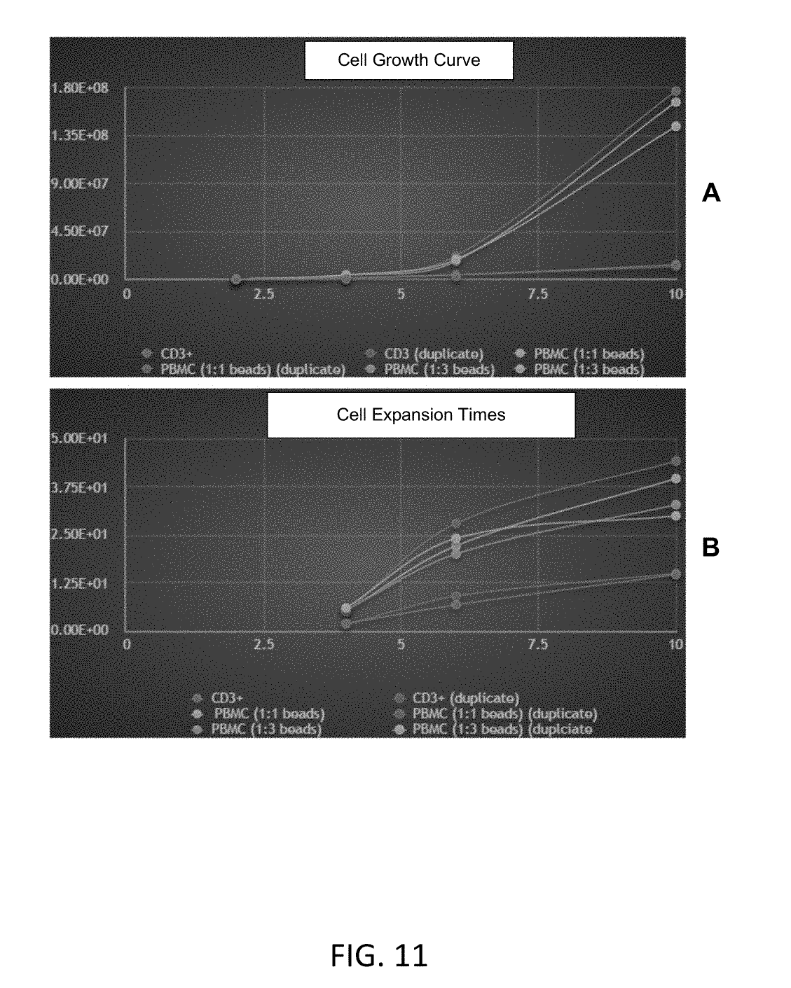

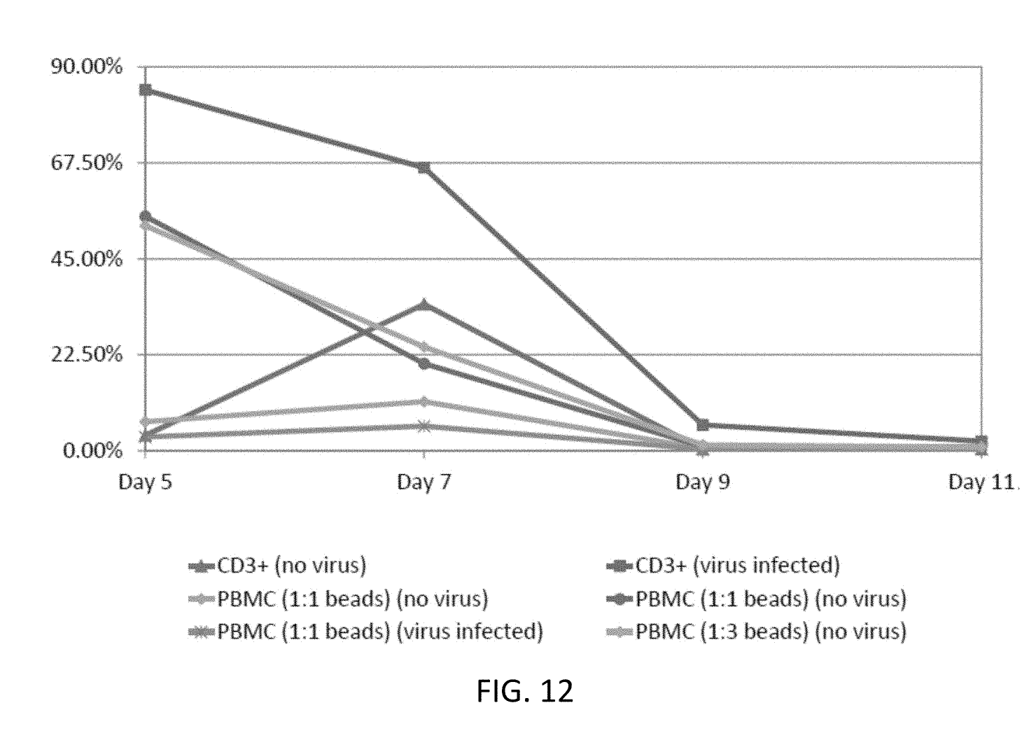

As flow cytometry analysis illustrated in FIG. 10, the protocol of the present disclosure isolated cell population consisted up to 98.48% of CD3+ cells; whereas samples derived from another method only achieved 48.43% CD3+ cells. In FIG. 11, the T cell proliferation rate using the protocol of the present disclosure isolated T cell was not superior to other methods. It is possible that cell-to-cell interaction within a more dynamic cell population promotes cell proliferation, and 1:3 bead stimulation is expected to proliferate faster. As illustrated in FIG. 12, T cells isolated using the protocol of the present disclosure demonstrated superior lentivector infectivity at the second day post-infection, and the percentage of CAR-T expressing cells remained higher until Day 9.

Sequence identifiers for various constructs are provided in table 2.

| SEQ ID NO: | Identifiers | Sequence |
|---|---|---|
| SEQ ID NO: 1 | CD19 | MPPPRLLFFLLFLTPMEVRPEEPLVVKVEEGDNAVLQCLKGTSDG |
| | antigen | PTQQLTWSRESPLKPFLKLSLGLPGLGIHMRPLAIWLFIFNVSQQ |
| | | MGGFYLCQPGPPSEKAWQPGWIVNVEGSGELFRWNVSDLGGL |
| | | GCGLKNRSSEGPSSPSGKLMSPKLYVWAKDRPEIWEGEPPCLPPR |
| | | DSLNQSLSQDLTMAPGSTLWLSCGVPPDSVSRGPLSWTHVHPK |
| | | GPKSLLSLELKDDRPARDMWVMETGLLLPRATAQDAGKYYCHRG |
| | | NLTMSFHLEITARPVLWHWLLRTGGWK |
| SEQ ID NO: 2 | CAR | atggccttaccagtgaccgccttgctcctgccgctggccttgctgctccacgccgcc |
| | | aggccggacatccagatgacacagactacatcctccctgtctgcctctctgggaga |
| | | cagagtcaccatcagttgcagggcaagtcaggacattagtaaatatttaaattggt |
| | | atcagcagaaaccagatggaactgttaaactcctgatctaccatacatcaagatta |
| | | cactcaggagtcccatcaaggttcagtggcagtgggtctggaacagattattctctc |
| | | accattagcaacctggagcaagaagatattgccacttacttttgccaacagggtaa |
| | | tacgcttccgtacacgttcggaggggggaccaagctggagatcacaggtggcggt |
| | | ggctcgggcggtggtgggtcgggtggcggcggatctgaggtgaaactgcaggagt |
| | | caggacctggcctggtggcgccctcacagagcctgtccgtcacatgcactgtctca |
| | | ggggtctcattacccgactatggtgtaagctggattcgccagcctccacgaaaggg |
| | | tctggagtggctgggagtaatatggggtagtgaaaccacatactataattcagctc |
| | | tcaaatccagactgaccatcatcaaggacaactccaagagccaagttttcttaaaa |
| | | atgaacagtctgcaaactgatgacacagccatttactactgtgccaaacattattac |
| | | tacggtggtagctatgctatggactactggggccaaggaacctcagtcaccgtctc |
| | | ctcaaccacgacgccagcgccgcgaccaccaacaccggcgcccaccatcgcgtcg |
| | | cagcccctgtccctgcgcccagaggcgtgccggccagcggcggggggcgcagtg |
| | | cacacgagggggctggacttcgcctgtgatatctacatctgggcgcccttggccgg |
| | | gacttgtggggtccttctcctgtcactggttatcaccctttactgcaaacggggcaga |
| | | aagaaactcctgtatatattcaaacaaccatttatgagaccagtacaaactactca |
| | | agaggaagatggctgtagctgccgatttccagaagaagaagaaggaggatgtga |
| | | actgagagtgaagttcagcaggagcgcagacgcccccgcgtacAagcagggcca |
| | | gaaccagctctataacgagctcaatctaggacgaagagaggagtacgatgttttgg |
| | | acaagagacgtggccgggaccctgagatggggggaaagccgagaaggaagaac |
| | | cctcaggaaggcctgtacaatgaactgcagaaagataagatggcggaggcctac |
| | | agtgagattgggatgaaaggcgagcgccggaggggcaaggggcacgatggcctt |
| | | taccagggtctcagtacagccaccaaggacacctacgacgcccttcacatgcagg |
| | | ccctgccccctcgctaa |

What is claimed is:

1. A method of selectively ex vivo activating a population of T cells for use in therapy, the method comprising:
contacting the population of T cells with an agent to activate the population of T cells that express a CAR to release IFN gamma, the agent comprising an extracellular domain of an antigen, the CAR comprising an extracellular domain, a transmembrane domain, an intracellular domain.

2. The method of claim 1, wherein the antigen comprises HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α2, IL-11 receptor a, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor

3. The method of claim 1, further comprising:
prior to contacting the population of T cells with the agent,
(a) contacting the population of T cells with an anti-CD3 antibody that is immobilized on a solid surface for a time period;
(b) transferring a nucleic acid sequence encoding the CAR to the contacted population of T cells; and
(c) removing the solid surface from the population of T cells after the transferring.

4. The method of claim 1, further comprising:
collecting peripheral blood mononuclear cells (PBMCs) from a subject; and
selecting CD3⁺ cells from the PBMCs.

5. The method of claim 1, wherein the selecting the CD3⁺ cells from the PBMCs comprises:
mixing the PBMCs with a group of antibodies to allow the group of antibodies to bind target cells, the group of antibodies not including CD3 antibodies; and
removing the target cells from the PBMCs to obtain a solution containing the CD3⁺ cells.

6. The method of claim 1, wherein the group of antibodies comprises CD14, CD15, CD16, CD19, CD34, CD36, CD56, CD123, and CD235a.

7. The method of claim 1, wherein the antigen comprises the amino acid sequence of SEQ ID NO: 1.

8. The method of claim 1, wherein the agent is attached to a surface.

9. The method of claim 8, wherein the surface comprises a bead.

10. The method of claim 9, wherein the bead is capable of activating the population of T cells expressing the CAR to release IFN gamma.

11. The method of claim 8, wherein the surface comprises a magnetic particle.

12. The method of claim 1 wherein the population of T cells is ex vivo expanded to a sufficient number for use in therapy.

13. The method of claim 12, wherein the population of T cells is expanded to at least 100-fold of the original T cell population.
